(19) United States
(12) Patent Application Publication
(10) Pub. No.: US 2006/0013842 A1
(43) Pub. Date: Jan. 19, 2006

(54) Natural mixture of long-chain fatty alcohols and long-chain fatty acids, its obtension from animal and vegetable waxes and its nutraceutical uses

(76) Inventors: John Roger Matkin, Exeter, CA (US); Sanyong Wang, Guangzhou (CN); Chunrong Li, Guangzhou (CN); Weng Huang, Guangzhou (CN)

Correspondence Address: Mitchell, Silberberg & Knupp, LLP, 11377 West Olympic Boulevard, Los Angeles, CA, 90064, US

(21) Appl. No.: 11/119,893

(22) Filed: May 3, 2005

Related U.S. Application Data
(60) Provisional application No. 60/588,304, filed on Jul. 15, 2004.

(51) Int. Cl. A61K 8/02

(52) U.S. Cl. 424/401

(57) ABSTRACT

This invention is related to the obtention of a new natural mixture composed of long chain fatty alcohols and long chain fatty acids. This mixture has a relative composition of long chain fatty alcohols and long chain fatty acids that is highly reproducible batch to batch and it is extracted from animal or vegetable wax. This mixture has specific therapeutic properties that support its use as an active component of dietary supplement formulations for reducing serum cholesterol levels (while not decreasing HDL-cholesterol levels) and therefore is effective treating Hypercholesterolemia and reducing the risk of coronary heart disease. This mixture also has been shown to improve male sexual activity. When used in conjunction with salicylic acid the mixture has other therapeutic effects such as inhibiting the atherosclerotic process, platelet hyperaggregability, inhibiting thrombosis and inhibiting inflammation.

### BACKGROUND OF THE INVENTION

This invention relates to a mixture of long chain fatty alcohols and long chain fatty acids (hereinafter called LCFA-FA), which are obtained from sugar cane wax, rice bran wax, bee's wax, insect wax or sorghum wax. The mixture from each wax shows a relative composition of each alcohol and acid that is highly reproducible batch to batch.

According to the invention, the mixture, of long chain fatty alcohols (hereinafter called LCFA) and long chain fatty acids (hereinafter called FA), has specific therapeutic properties. The mixture can be used as an active component in nutraceutical formulations for reducing serum cholesterol levels and other therapeutic uses.

Sugar cane wax and its natural source, mud, have always been a matter of interest, not only because of their industrial application, but also because of their chemical composition. The amount of wax in sugar cane ranges between 0.1% and 0.3%, depending on its variety and growing conditions.

During the agro industrial process, approximately 40% of the sugar cane wax content is distributed within the juice; the remaining wax is lost in the bagasse. From this 40%, 95% of it is absorbed by the mud, from which raw wax is obtained. This wax is made up of esters, aldehydes, ketones, hydrocarbons, fatty acids and free alcohols, the amount of each depending on the variety and origin of the sugar cane plant and the technology used to obtain the wax.

Several authors, to learn about their composition and main features, have studied the long chain fatty alcohols obtained from sugar cane byproducts. The obtention of different groups of compounds from all kinds of waxes has been reported (J. A. Lamberton.et al., 1959; Australian Journal of Chemistry 13, 261-268 and Horn A. and Martic J. S., 1957; Journal of Science Food and Agriculture 10, 571). The authors suggest a method for obtaining long chain fatty alcohols from sugar cane cuticular wax based on homogeneous saponification with alcoholic potassium hydroxide, followed by esterification of the unsaponifiable material and further molecular distillation.

Another method has been reported to isolate long chain fatty alcohols through a high efficiency high vacuum column. High vacuum wax distillation is used for the chemical isolation of carbonylic compounds and the extraction of the remaining wax uses petrol ether. The solvent evaporates and the remaining content is acetylated for its further isolation through alumina chromatography. Finally, through alkaline hydrolysis, alcohols are obtained and then recrystallized in ethanol, showing a fusion point ranging between 80 and 82 degrees C.

A procedure for producing long chain fatty alcohols from animal and vegetable wax, is based on the saponification of the fatty esters followed by the extraction of alcohol mixtures through a fluid in sub- and supercritical states of CO·sub·2, pressures ranging from 60-300 kg/cm·sub·2 and temperatures between 25 and 100 degrees C. using adequate solvents, showing that depending on the solubility and at low temperature and pressure changes, selective extraction can be carried out. According to this procedure applied to the sugar cane wax, it is possible to obtain 5% long chain fatty acids.

Another project (Inada S., Furukawa K., Masui T., Honda K., Ogasawara J. and Tsubikamoto G.; 1986; Process for recovering primary normal aliphatic higher alcohols JP 62-087537) proposed a very similar extraction method applied to waxes that are based on fluids in sub- and supercritical states of CO·sub·2 with ethylene. The separation of organic compounds from their mixtures by means of fluids in sub- and supercritical states is also described.

From the analytical point of view, all these are valuable methods, but high scale implementation is hindered by the use of column chromatography and molecular distillation, which are all not economical procedures.

There are other mixtures of fatty acids that have reported biological properties. Such is the case reported in U.S. Pat. No. 5,284,873 in which a pharmaceutical composition for combating prostate affections, in which the active principle is a fraction of fatty acids obtained from the fruits of Sabal Serrulata, with oleic acid as the main one, followed by lauric and palmitic acids, with eicosenoic acid having the greater number of carbon atoms. In U.S. Pat. No. 5,502,045, a method is disclosed to reduce the cholesterol levels in serum using an ester formed by a-sitostanol and two fatty acids from 2 to 22 carbon atoms. It also discloses a method of formation of such esters, having the effective daily dose of 0.2 to 20 grams of the ester.

In U.S. Pat. No. 5,444,054 a method is reported for treating ulcerative colitis in which one of the fractions used is an oil that contains certain unsaturated fatty acids from 18 to 22 carbon atoms. U.S. Pat. Nos. 4,505,933 and 4,687,783 disclose mixtures of fatty aldehydes derived from fatty acids, for the treatment of patients with multiple sclerosis, as well as neurological and dermatological diseases. The inventors disclose a daily dose of 100-400 mg/kg of body weight of the patients.

In the last decade, numerous patents have appeared, that discuss omega-3-poly-unsaturated fatty acids and their effect on serum cholesterol and blood platelet aggregation. Such is the case in U.S. Pat. No. 4,526,902, that discusses a pharmaceutical composition for the treatment of thrombo-embolic conditions in which the active principles are unsaturated eicosapentaenoic and docohexanoic fatty acids together with linoleic acid, linolenic acid and its derivatives. U.S. Pat. No. 5,502,077 discloses a composition of fatty acids for the treatment of prophylaxis of multiple risk factors for cardiovascular diseases, where 80% of the weight corresponds to omega-3 fatty acids, its salts or derivatives thereof, with the main ones being eicosapentaenoic and docohexanoic acids. EPO patent 0 422 490 A2 discloses a pharmaceutical composition for inhibiting the absorption of cholesterol, containing triglycerides formed, preferably, by a mixture of saturated fatty acids from 20 to 24 carbon atoms, but they should be administered in a daily does of 2 to 10 grams. The Japanese patents, 55092316 A and 56115736 A of Tokiwa Shizeru et al, report the demonstration, as well as the isolation and purification, of an agent for diminishing cholesterol composed of a mixture of highly unsaturated fatty acids, especially eicosatrienoic and docosatertraenoic acids. Other Japanese patents (publication #1290625 A, 02053724 A, 02243622 A and 04169524 A) claim different pharmaceutical formulations, such as an improver of cerebral function or for the treatment of degenerative disease, or for lowering cholesterol in blood and for having serum lipid-improving activity in which mixtures of fatty acids, especially eicosapentaenoic and docosahexanoic acids are present as active principles with a daily dose that varies between 500 mg/kg of body weight and 0.5-30 grams.

The procedure for the obtention of the mixture of fatty acids in U.S. Pat. No. 6,486,204 patent is based on a homogeneous saponification of sugar cane wax with concentrated solutions of alkaline and earth-alkaline hydroxides, especially those of low molecular weight and more especially those of sodium, potassium and calcium. The concentration of the alkaline solution must be such that the ratio in weight of the corresponding hydroxide with that of the wax to be processed must be more than 5%, specifically between 8 and 25% and more specifically between 15 and 25%. The saponification process lasts for a period exceeding 30 minutes and more specifically between one and five hours. The solid, obtained in this step, is processed using a conventional solid-liquid extractor, where the fatty acids, in salt form, are isolated from the rest of the components by extracting these components using the appropriate organic solvent. The mixture of fatty acids in salt form is purified by successive recrystallizations in appropriate organic solvents or in aqueous solutions.

In the final step of this process, the fatty acids are regenerated using an acid solution that could be prepared using mineral acids and/or organic acids. The yield of fatty acids is between 10 and 40%, while the purity of the fatty acids is in the general range between 85 and 100%, more specifically, between 90 and 99%, determined using gas chromatography and/or volumetric chemical analysis.

U.S. Pat. No. 5,856,316 discloses a composition of long chain fatty alcohols from sugar cane wax used as a hypocholesterolemic and against hypercholesterolemia type II, as an antiplatelet, anti-thrombotic and anti-ischemic agent.

U.S. Pat. No. 6,486,205 claims a composition of fatty acids also used as a hypocholesterolemic and against hypercholesterolemia type II, as an antiplatelet, anti-thrombotic and anti-ischemic agent.

One of the objects of the present invention is to isolate and purify the natural mixture of long chain fatty alcohols and long chain fatty acids, specifically the mixture of 60-95% by weight, long chain fatty alcohols and 5-40% by weight, long chain fatty acids. Other objects of this invention are to use this natural mixture of long chain fatty alcohols and long chain fatty acids in relatively low doses, as a component of nutraceutical, dietary supplement, food and beverage formulations used for their cholesterol-lowering, cardiovascular and other health benefits.

### SUMMARY OF THE INVENTION

The composition obtained in the present invention is a mixture of long chain fatty alcohols and long chain fatty acids. Table 1 reports the qualitative and quantitative composition of this fatty acid composition in the long chain fatty alcohol, long chain fatty acids, mixture.

TABLE 1
Qualitative and Quantitative Composition of
FA in LCFA-FA from Sugar Cane Wax

| | Percentage in the |
|---|---|
| Component | mixture of LCFA-FA (weight) |
| 1-tetracosanoic acid | 0.0-2.0% |
| 1-hexacosanoic acid | 0.1-5.0% |
| 1-octacosanoic acid | 2.0-25% |
| 1-nonacosanoic acid | 0.0-2.0% |
| 1-triacontanoic acid | 0.1-6.0% |
| 1-dotriacontanoic acid | 0.0-3.0% |
| 1-tetratriacontanoic acid | 0.0-3.0% |
| 1-hexatriacontanoic acid | 0.0-1.0% |

The daily dose of long chain fatty alcohols and long chain fatty acids mixture to be used for reducing serum cholesterol levels is between 1 and 50 mg per day and the most appropriate route of administration is oral solid dosage form, such as tablets, or capsules, or as an addition to food or beverage products.

The procedure for the obtention of this mixture of long chain fatty alcohols and long chain fatty acids, in the present invention, is based on an ester-exchange reaction of vegetable or animal waxes with sodium ethylate (or methylate) solution. The ester-exchange process lasts for a period more than one hour and more specifically between three and six hours. The solvent evaporates and the remaining content is processed using molecular distillation to obtain the mixture of long chain fatty alcohols and the ethyl (or methyl) ester of fatty acids. The mixture of long chain fatty alcohols and the ethyl (or methyl) ester of fatty acids are saponified and the mixture is regenerated using mineral acids. The mixture of long chain fatty alcohols and long chain fatty acids can also be obtained using other methods, as well.

### DETAILED DESCRIPTION OF THE INVENTION

The invention is described in detail by the following representative examples for the production of the long chain fatty alcohols and long chain fatty acids mixture.

### EXAMPLE 1

One thousand (1000) grams of refined sugar cane wax was dissolved in 20 liters of anhydrous ethanol and 10 grams sodium ethylate was added. The ester-exchange reaction of the mixture was heated under reflux for three hours.

The ethanol was then removed under vacuum. The mixture of long chain fatty alcohols and the ethyl ester of fatty acids (400 grams) were obtained by molecular distillation. Four hundred (400) grams of long chain fatty alcohols and the ethyl ester of fatty acids were melted at 100-110 degrees C., to which 10 grams of sodium hydroxide, dissolved in 15 ml of water, was added. The hydrolysis process was maintained for a period of four hours, stirring periodically. The mixture of long chain fatty alcohols-fatty acids is regenerated by treatment with concentrated sulfuric acid. Three hundred eight (380) grams of this mixture of long chain fatty alcohols (86.4%) and fatty acids (10.3%) were obtained with a purity of 96.7%.

### EXAMPLE 2

Ten (10) kilograms of raw sugar cane wax was dissolved in 200 liters anhydrous ethanol and 200 grams sodium ethylate was added. The reaction of the mixture was heated under reflux for four hours. The ethanol was then removed under vacuum. The mixture of long chain fatty alcohols and the ethyl ester of fatty acids (3 kilograms) were obtained by molecular distillation. Three (3) kilograms of the mixture of long chain fatty alcohols and the ethyl ester were melted at 100-110 degrees C., to which 50 grams of sodium hydroxide dissolved in 80 ml of water, was added. The hydrolysis process was maintained for a period of six hours with periodic stirring. The mixture of long chain fatty alcohols-fatty acids was regenerated by treatment with concentrated sulfuric acid. Two thousand eight hundred (2800) grams of this mixture was obtained with a purity of 98%. Table 2 records the qualitative and quantitative composition of the long chain fatty alcohols-fatty acids.

TABLE 2
Qualitative and Quantitative Composition of
LCFA-FA Obtained from Sugar Cane Wax

| | Percentage of Each Alcohol |
|---|---|
| Component | or Acid in LCFA-FA (weight) |
| 1-tetracosanol | 1.5% |
| 1-hexacosanol | 9.5% |
| 1-heptacosanol | 1.1% |
| 1-octacosanol | 58.8% |
| 1-nonacosanol | 2.3% |
| 1-triacontanol | 7.6% |
| 1-dotriacontanol | 2.0% |
| 1-tetratriacontanol | 1.0% |
| 1-hexacosanoic acid | 0.15% |
| 1-octacosanoic acid | 9.6% |
| 1-nonaconsanoic acid | 0.15% |
| 1-triacontanoic acid | 1.8% |
| 1-dotriacontanoic acid | 0.75% |
| 1-tetratriacontanoic acid | 0.65% |
| 1-hextriacontanoic acid | 0.10% |

### EXAMPLE 3

Eight (8) kilograms of rice bran wax was dissolved in 200 liters anhydrous ethanol and 200 grams sodium ethoxide was added. The reaction of the mixture was heated under reflux for five hours. The ethanol was then removed under vacuum. The mixture of long chain fatty alcohols and the ethyl ester of fatty acids (3.0 kilograms) were obtained by molecular distillation. Three (3) kilograms of the mixture of long chain fatty alcohols and the ethyl ester were melted at 100-110 degrees C., to which 100 grams of sodium hydroxide dissolved in 150 ml of water, was added. The hydrolysis process was maintained for a period of six hours with periodic stirring. The mixture of long chain fatty alcohols-fatty acids was regenerated by treatment with concentrated sulfuric acid. Two thousand four hundred (2400) grams of this mixture was obtained with a purity of 98%.

Table 3 records the qualitative and quantitative composition of the long chain fatty alcohols-fatty acids.

TABLE 3
Qualitative and Quantitative Composition of
LCFA-FA Obtained from Rice Bran Wax

| | Percentage of Each Alcohol |
|---|---|
| Component | or Acid in LCFA-FA (weight) |
| 1-tetracosanol | 6.0% |
| 1-hexacosanol | 16.6% |
| 1-heptacosanol | 0.5% |
| 1-octacosanol | 18.5% |
| 1-nonacosanol | 0.6% |
| 1-triacontanol | 25.6% |
| 1-dotriacontanol | 10.8% |
| 1-tetratriacontanol | 3.4% |
| 1-docosanoic acid | 4.5% |
| 1-tetracosanoic acid | 10.2% |
| 1-hexacosanoic acid | 0.98% |
| 1-octacosanoic acid | 0.16% |
| 1-triacontanoic acid | 0.16% |

### EXAMPLE 4

One kilogram of bee's wax was dissolved in 200 liters anhydrous ethanol and 200 grams sodium ethoxide was added. The reaction of the mixture was heated under reflux for five hours. The ethanol was then removed under vacuum. The mixture of long chain fatty alcohols and the ethyl ester of fatty acids (300 grams) were obtained by molecular distillation. Three hundred (300) grams of the mixture of long chain fatty alcohols and the ethyl ester were melted at 100-110 degrees C., to which five grams of sodium hydroxide dissolved in 10 ml of water, was added. The hydrolysis process was maintained for a period of six hours with periodic stirring. The mixture of long chain fatty alcohols-fatty acids was regenerated by treatment with concentrated sulfuric acid. Two hundred seventy (270) grams of this mixture was obtained with a purity of 98%. Table 4 records the qualitative and quantitative composition of the long chain fatty alcohols-fatty acids.

TABLE 4
Qualitative and Quantitative Composition of
LCFA-FA Obtained from Bee's Wax

| | Percentage of Each Alcohol |
|---|---|
| Component | or Acid in LCFA-FA (weight) |
| 1-tetracosanol | 1.2% |
| 1-hexacosanol | 3.8% |
| 1-heptacosanol | 0.2% |
| 1-octacosanol | 18.9% |
| 1-nonacosanol | 0.35% |
| 1-triacontanol | 54.5% |
| 1-dotriacontanol | 6.3% |
| 1-tetratriacontanol | 2.0% |
| 1-tetracosanoic acid | 0.15% |
| 1-hexacosanoic acid | 0.30% |
| 1-octacosanoic acid | 1.1% |
| 1-triacontanoic acid | 5.4% |
| 1-dotriacontanoic acid | 2.5% |
| 1-tetratriacontanoic acid | 1.3% |

### EXAMPLE 5

Eight (8) kilograms of insect wax was dissolved in 200 liters anhydrous ethanol and 200 grams sodium ethoxide was added. The reaction of the mixture was heated under reflux for five hours. The ethanol was then removed under vacuum. The mixture of long chain fatty alcohols and the ethyl ester of fatty acids (4 kilograms) were obtained by molecular distillation. Four (4) kilograms of the mixture of long chain fatty alcohols and the ethyl ester were melted at 100-110 degrees C., to which 100 grams of sodium hydroxide dissolved in 150 ml of water, was added. The hydrolysis process was maintained for a period of six hours with periodic stirring. The mixture of long chain fatty alcohols-fatty acids was regenerated by treatment with concentrated sulfuric acid. Three thousand seven hundred (3700) grams of this mixture was obtained with a purity of 99%. Table 5 records the qualitative and quantitative composition of the long chain fatty alcohols-fatty acids.

TABLE 5
Qualitative and Quantitative Composition of
LCFA-FA Obtained from Insect Wax

| | Percentage of Each Alcohol |
|---|---|
| Component | or Acid in LCFA-FA (weight) |
| 1-tetracosanol | 4.5% |
| 1-hexacosanol | 42.0% |
| 1-heptacosanol | 0.2% |
| 1-octacosanol | 18.5% |
| 1-triacontanol | 2.1% |
| 1-tetracosanoic acid | 4.5% |
| 1-hexacosanoic acid | 17.1% |
| 1-octacosanoic acid | 8.1% |
| 1-triacontanoic acid | 1.0% |

### EXAMPLE 6

One thousand (1000) grams of refined sorghum wax was dissolved in 20 liters of anhydrous ethanol and 10 grams sodium ethylate was added. The ester-exchange reaction of the mixture was heated under reflux for three hours. The ethanol was then removed under vacuum. The mixture of long chain fatty alcohols and the ethyl ester of fatty acids (330 grams) were obtained by molecular distillation. Four hundred (400) grams of long chain fatty alcohols and the ethyl ester of fatty acids were melted at 100-110 degrees C., to which 10 grams of sodium hydroxide, dissolved in 150 ml of water, was added. The mixture of long chain fatty alcohol-fatty acids was regenerated by treatment with concentrated sulfuric acid. Two hundred eighty (280) grams of this mixture of long chain fatty alcohols (87.0%) and fatty acids (8.1%) were obtained with a purity of 95.1%. Table 6 records the qualitative and quantitative composition of the long chain fatty alcohols-fatty acids.

TABLE 6
Qualitative and Quantitative Composition of
LCFA-FA Obtained from Sorghum Wax

| | Percentage of Each Alcohol |
|---|---|
| Component | or Acid in LCFA-FA (weight) |
| 1-tetracosanol | 0.4% |
| 1-hexacosanol | 5.1% |
| 1-heptacosanol | 0.6% |
| 1-octacosanol | 41.2% |
| 1-nonacosanol | 1.0% |
| 1-triacontanol | 35.6% |
| 1-dotriacontanol | 2.8% |
| 1-tetratriacontanol | 0.4% |
| 1-hexacosanoic acid | 1.5% |
| 1-octacosanoic acid | 3.6% |
| 1-triacontanoic acid | 3.0% |

The Examples show the formulation of a mixture of long chain fatty alcohols from 24 to 34 carbon atoms and long chain fatty acids from 22 to 38 carbon atoms, wherein the long chain fatty alcohols are present from 60-95% by weight and the long chain fatty acids are present from 5-40% by weight.

The long chain fatty alcohol component contains the following components:

| | | |
|---|---|---|
| 1-tetracosanol | 0.1-10% | by weight |
| 1-hexacosanol | 3.0-60% | by weight |
| 1-heptacosanol | 0.0-3.0% | by weight |
| 1-octacosanol | 6.0-80.0% | by weight |
| 1-nonacosanol | 0.0-3.0% | by weight |
| 1-triacontanol | 2.0-65.0% | by weight |
| 1-dotriacontanol | 0.0-20.0% | by weight |
| 1-tetratriacontanol | 0.0-12.0% | by weight |

The long chain fatty acid component contains the following components:

| | |
|---|---|
| 1-docasanoic acid | 0.0-10.0% |
| 1-tetracosanoic acid | 0.0-10.0% |
| 1-hexacosanoic acid | 0.1-24.0% |
| 1-octacosanoic acid | 0.0-25.0% |
| 1-nonacosanoic acid | 0.0-2.0% |
| 1-triacontanoic acid | 0.0-15.0% |
| 1-dotriacantanoic acid | 0.0-80% |
| 1-tetratriacontanoic acid | 0.0-4.0% |
| 1-hexatriacontanoic acid | 0.0-1.0% |

The mixture of long chain fatty acid alcohols and long chain fatty acids obtained from sugar can wax, rice bran wax, bee's wax, insect wax or sorghum wax is produced by an ester-exchange reaction of the above wax in sodium ethylate (or methylate) solution; molecular distillation of long chain fatty alcohols and ethyl (or methyl) ester of long chain fatty acids; saponifying the mixture of long chain fatty alcohols and ethyl (or methyl) ester of long chain fatty acids; and regenerating the above mixture using mineral acids.

The therapeutically beneficial dosage is administered at an amount of 1 to 50 mg per day.

This mixture has specific therapeutic properties that support its use as an active component of dietary supplement formulations for reducing serum cholesterol levels, while not decreasing HDL-cholesterol levels, and therefore it is effective in treating hypercholesterolemia and reducing the risk of coronary heart disease.

The mixture can be used in conjunction with salicylic acid to inhibit the atheroscherotic process, inhibiting thrombosis and inhibiting inflammation.

What is claimed is:

1. A therapeutic mixture comprising long chain fatty alcohols having from 24-34 carbon atoms and long chain fatty acids having from 22-38 carbon atoms wherein the long chain fatty alcohols (C24 C34) is 60-95% by weight and the long chain fatty acids (C22 C38) are 5-40% by weight.

2. The therapeutic mixture of long chain fatty alcohols and long chain fatty acids from sugar cane wax according to claim 1, comprising:

| | |
|---|---|
| 1-tetracosanol | 0.1-7.0% |
| 1-hexacosanol | 3.0-15.0% |
| 1-heptacosanol | 0.1-3.0% |
| 1-octacosanol | 50.0-80.0% |
| 1-nonacosanol | 0.0-3.0% |
| 1-triacontanol | 3.0-19.0% |
| 1-dotriacontanol | 0.2-9.0% |
| 1-tetratriacontanol | 0.0-3.0% |
| 1-tetracosanic acid | 0.0-2.0% |
| 1-hexacosanoic acid | 0.1-5.0% |
| 1-octacosanoic acid | 2.0-25.0% |
| 1-nonacosanoic acid | 0.0-2.0% |
| 1-triacontanoic acid | 0.1-6.0% |
| 1-dotriacontanoic acid | 0.0-3.0% |
| 1-tetratriacontanoic acid | 0.0-3.0% |
| 1-hexatriacontanoic acid | 0.0-1.0% |

3. The therapeutic mixture of long chain fatty alcohols and long chain fatty acids from rice bran wax according to claim 1, comprising:

| | |
|---|---|
| 1-tetracosanol | 0.1-10.0% |
| 1-hexacosanol | 3.0-20.00% |
| 1-heptacosanol | 0.0-3.0% |
| 1-octacosanol | 12.0-40.0% |
| 1-nonacosanol | 0.0-3.0% |
| 1-triacontanol | 12.0-40.0% |
| 1-dotriacontanol | 3.0-20.0% |
| 1-tetratriacontanol | 0.1-6.0% |
| 1-docosanoic acid | 1.0-10.0% |
| 1-tetracosanoic acid | 3.0-25.0% |
| 1-hexacosanoic acid | 0.1-2.0% |
| 1-octacosanoic acid | 0.0-1.0% |
| 1-triacontanoic acid | 0.0-1.0% |

4. The therapeutic mixture of long chain fatty alcohols and long chain fatty acids from bee's wax according to claim 1, comprising:

| | |
|---|---|
| 1-tetracosanol | 0.3-10.0% |
| 1-hexacosanol | 5.0-20.0% |
| 1-heptacosanol | 0.1-3.0% |
| 1-octacosanol | 6.0-30.0% |
| 1-nonacosanol | 0.0-3.0% |
| 1-triacontanol | 25.0-65.0% |
| 1-dotriacontanol | 2.0-18.0% |
| 1-tetratriacontanol | 0.1-12.0% |
| 1-tetracosanoic acid | 0.0-2.0% |
| 1-hexacosanoic acid | 0.0-2.0% |
| 1-octacosanoic acid | 0.2-4.0% |
| 1-triacontanoic acid | 2.5-15.0% |
| 1-dotriacontanoic acid | 0.4-8.0% |
| 1-tetratriacontanoic acid | 0.1-4.0% |

5. The therapeutic mixture of long chain fatty alcohols and long chain fatty acids from insect wax according to claim 1, comprising:

| | |
|---|---|
| 1-tetracosanol | 2.0-10.0% |
| 1-hexacosanol | 35.0-60.0% |
| 1-heptacosanol | 0.0-3.0% |
| 1-octacosanol | 15.0-40.0% |
| 1-nonacosanol | 0.0-3.0% |
| 1-triacontanol | 2.0-10.0% |
| 1-tetracosanoic acid | 0.1-7.0% |
| 1-hexacosanoic acid | 2.5-24.0% |
| 1-octacosanoic acid | 0.5-12.0% |
| 1-triacontanoic acid | 0.0-3.0% |

6. The therapeutic mixture of long chain fatty alcohols and long chain fatty acids from sorghum wax according to claim 1, comprising:

| | |
|---|---|
| 1-tetracosanol | 0.0-3.0% |
| 1-hexacosanol | 4.0-7.0% |
| 1-heptacosanol | 0.0-1.0% |
| 1-octacosanol | 30.0-48.0% |
| 1-nonacosanol | 0.1-2.0% |
| 1-triacontanol | 25.0-40.0% |
| 1-dotriacontanol | 0.5-4.0% |
| 1-tetratriacontanol | 0.0-2.0% |
| 1-hexacosanoic acid | 0.5-2.0% |
| 1-octacosanoic acid | 2.0-4.0% |
| 1-triacontanoic acid | 2.0-4.0% |
| 1-dotriacontanoic acid | 0.1-2.0% |

7. A method for producing a nutraceutically beneficial mixture of long chain fatty alcohols and long chain fatty acids from sugar cane wax, rice bran wax, bee's wax, insect wax or sorghum wax, comprising the following steps of:
(a) ester-exchange reaction of the above wax in sodium ethylate (or methylate) solution;
(b) molecular distillation of long chain fatty alcohols and ethyl (or methyl) ester of long chain fatty acids;
(c) saponifying the mixture of long chain fatty alcohols and ethyl (or methyl) ester of long chain fatty acids;
and
(d) regenerating the above mixture using mineral acids.

8. A method for reducing serum cholesterol levels in humans by administering a mixture containing approximately 60% to 95% by weight of long chain fatty alcohols, and approximately 5% to 40% by weight of long chain fatty acids.

9. The therapeutic mixture of long chain fatty alcohols and long chain fatty acids from sugar cane wax according to claim 8, comprising:

| | |
|---|---|
| 1-tetracosanol | 0.1-7.0% |
| 1-hexacosanol | 3.0-15.0% |
| 1-heptacosanol | 0.1-3.0% |
| 1-octacosanol | 50.0-80.0% |
| 1-nonacosanol | 0.0-3.0% |
| 1-triacontanol | 3.0-19.0% |
| 1-dotriacontanol | 0.2-9.0% |
| 1-tetratriacontanol | 0.0-3.0% |
| 1-tetracosanic acid | 0.0-2.0% |
| 1-hexacosanoic acid | 0.1-5.0% |
| 1-octacosanoic acid | 2.0-25.0% |
| 1-nonacosanoic acid | 0.0-2.0% |
| 1-triacontanoic acid | 0.1-6.0% |
| 1-dotriacontanoic acid | 0.0-3.0% |
| 1-tetratriacontanoic acid | 0.0-3.0% |
| 1-hexatriacontanoic acid | 0.0-1.0% |

10. The therapeutic mixture of long chain fatty alcohols and long chain fatty acids from rice bran wax according to claim 8, comprising:

| | |
|---|---|
| 1-tetracosanol | 0.1-10.0% |
| 1-hexacosanol | 3.0-20.00% |
| 1-heptacosanol | 0.0-3.0% |
| 1-octacosanol | 12.0-40.0% |
| 1-nonacosanol | 0.0-3.0% |
| 1-triacontanol | 12.0-40.0% |
| 1-dotriacontanol | 3.0-20.0% |
| 1-tetratriacontanol | 0.1-6.0% |
| 1-docosanoic acid | 1.0-10.0% |
| 1-tetracosanoic acid | 3.0-25.0% |
| 1-hexacosanoic acid | 0.1-2.0% |
| 1-octacosanoic acid | 0.0-1.0% |
| 1-triacontanoic acid | 0.0-1.0% |

11. The therapeutic mixture of long chain fatty alcohols and long chain fatty acids from bee's wax according to claim 8, comprising:

| | |
|---|---|
| 1-tetracosanol | 0.3-10.0% |
| 1-hexacosanol | 5.0-20.0% |
| 1-heptacosanol | 0.1-3.0% |
| 1-octacosanol | 6.0-30.0% |
| 1-nonacosanol | 0.0-3.0% |
| 1-triacontanol | 25.0-65.0% |
| 1-dotriacontanol | 2.0-18.0% |
| 1-tetratriacontanol | 0.1-12.0% |
| 1-tetracosanoic acid | 0.0-2.0% |
| 1-hexacosanoic acid | 0.0-2.0% |
| 1-octacosanoic acid | 0.2-4.0% |
| 1-triacontanoic acid | 2.5-15.0% |
| 1-dotriacontanoic acid | 0.4-8.0% |
| 1-tetratriacontanoic acid | 0.1-4.0% |

12. The therapeutic mixture of long chain fatty alcohols and long chain fatty acids from insect wax according to claim 8, comprising:

| | |
|---|---|
| 1-tetracosanol | 2.0-10.0% |
| 1-hexacosanol | 35.0-60.0% |
| 1-heptacosanol | 0.0-3.0% |
| 1-octacosanol | 15.0-40.0% |
| 1-nonacosanol | 0.0-3.0% |
| 1-triacontanol | 2.0-10.0% |
| 1-tetracosanoic acid | 0.1-7.0% |
| 1-hexacosanoic acid | 2.5-24.0% |
| 1-octacosanoic acid | 0.5-12.0% |
| 1-triacontanoic acid | 0.0-3.0% |

13. The therapeutic mixture of long chain fatty alcohols and long chain fatty acids from sorghum wax according to claim 8, comprising:

| | |
|---|---|
| 1-tetracosanol | 0.0-3.0% |
| 1-hexacosanol | 4.0-7.0% |
| 1-heptacosanol | 0.0-1.0% |
| 1-octacosanol | 30.0-48.0% |
| 1-nonacosanol | 0.1-2.0% |
| 1-triacontanol | 25.0-40.0% |
| 1-dotriacontanol | 0.5-4.0% |
| 1-tetratriacontanol | 0.0-2.0% |
| 1-hexacosanoic acid | 0.5-2.0% |
| 1-octacosanoic acid | 2.0-4.0% |
| 1-triacontanoic acid | 2.0-4.0% |
| 1-dotriacontanoic acid | 0.1-2.0% |

14. The method of claim 8, said mixture derived from sugar cane wax, rice bran wax, bee's wax, insect wax or sorghum wax.

15. The method of according to claim 8, wherein the therapeutically beneficial dosage of said mixture ranges between 1 and 50 mg per day.

16. The method of claim 8, wherein the mixture further includes salicylic acid.
